(19) United States
(12) Patent Application Publication
(10) Pub. No.: US 2020/0030002 A1
(43) Pub. Date: Jan. 30, 2020

(54) INSTRUMENTS AND METHODS FOR MYOMA RETRACTION

(71) Applicant: COVIDIEN LP, Mansfield, MA (US)

(72) Inventors: Eric G. RAPP, Hauppauge, NY (US); Nikolai D. BEGG, Wellesley, MA (US); Tejas S. INAMDAR, San Francisco, CA (US)

(21) Appl. No.: 16/514,336

(22) Filed: Jul. 17, 2019

Related U.S. Application Data
(60) Provisional application No. 62/702,399, filed on Jul. 24, 2018.

(51) Int. Cl. A61B 17/42

(52) U.S. Cl. CPC A61B 17/4241; A61B 2017/00349

(57) ABSTRACT

A retraction instrument includes a tube, an actuator and a first coil. The tube defines a passage therethrough. The actuator has a handle that has a shaft extending distally therefrom through the passage of the tube. The first coil is disposed adjacent a distal end of the shaft. The first coil is configured to secure the shaft to tissue. A method of retracting tissue within a body cavity is also disclosed. The method includes inserting a retraction instrument into the body cavity of a patient, engaging tissue within the body cavity with a first coil of the retraction instrument to secure the retraction instrument to the tissue, and retracting the tissue with the retraction instrument.

### CROSS-REFERENCE TO RELATED APPLICATIONS

This application claims the benefit of, and priority to, U.S. Provisional Patent Application Ser. No. 62/702,399, filed on Jul. 24, 2018, the contents of which are incorporated herein by reference.

### BACKGROUND

### 1. Technical Field

The present disclosure relates generally to surgery. More particularly, the present disclosure relates to methods, devices, and systems for retracting fibroids.

### 2. Discussion of Related Art

During laparoscopic procedures, large segments of tissue and/or organs may need to be maneuvered, retracted, and/or removed from the body. For example, during laparoscopic myomectomies, fibroids may need to be retracted and/or removed from the body.

Fibroids are benign tumors of the uterine myometria, i.e., muscle, and are the most common tumor of the female pelvis. Fibroid tumors affect up to 30% of women of childbearing age and can cause significant symptoms such as discomfort, pelvic pain, menorrhagia, pressure, anemia, compression, infertility, and miscarriage. Fibroids may be located in the myometrium, adjacent to the endometrium (i.e., submucosal), or in the outer layer of the uterus (i.e., subserosal). Most commonly, fibroids are a smooth muscle overgrowth that arise within the walls of the myometrium and can grow to be several centimeters in diameter.

Surgical myomectomy is a laparoscopic surgical procedure performed by a surgeon to spare the tissue of the uterus. Unfortunately, due to the limited spatial access through the uterus, laparoscopic myomectomy remains technically challenging. A surgeon must access the uterus through a small incision and dissect the fibroid from the uterine wall with limited access and with limited directions of traction of tissue. Moreover, once the fibroid is dissected from the uterine wall the fibroid must be morcellated to remove the fibroid through the incision.

### SUMMARY

There is a continuing need for improved instruments and methods for retracting and dissecting fibroids during myomectomy procedures including laparoscopic myomectomy.

In an aspect of the present disclosure, a retraction instrument includes a tube, an actuator and a first coil. The tube defines a passage therethrough. The actuator has a handle that has a shaft extending distally therefrom through the passage of the tube. The first coil is disposed adjacent a distal end of the shaft. The first coil is configured to secure the shaft to tissue.

In aspects, the first coil extends distally from the distal end of the shaft. The first coil may include a straight configuration in which the first coil is substantially linear and a coiled configuration in which the first coil is substantially non-linear and configured to secure the first coil within tissue. The first coil may be configured to transition from the straight configuration to the coiled configuration as the first coil is inserted into tissue.

In some aspects, the shaft has a retracted position in which the first coil is disposed within the tube and an extended position in which the first coil is positioned distal of the tube.

In certain aspects, the retraction instrument includes a second coil that is secured to a distal portion of the tube and extends distally from the distal portion of the tube. The first coil may be wound in a first direction, e.g., clockwise, and the second coil may be wound in a second opposite direction, e.g., counter-clockwise. The first coil may have a first diameter and the second coil may have a second diameter which is greater than the first diameter. The first coil may have a substantially constant diameter.

In particular aspects, the retraction instrument includes a rod that extends through the actuator to a tip that is disposed distal of the distal end of the shaft. The first coil may include a leading end that is secured to the tip and a trailing end that is secured to the shaft. The first coil may wind about the shaft between the leading and trailing ends. The first coil may have a contracted configuration in which the first coil defines a first diameter and an expanded configuration in which the first coil defines a second diameter greater than the first diameter. The rod may be rotatable relative to the shaft to transition the first coil between the contracted and expanded configuration. The first diameter may be less than a diameter of the passage.

In another aspect of the present disclosure, a method of retracting tissue within a body cavity is also disclosed. The method includes inserting a retraction instrument into the body cavity of a patient, engaging tissue within the body cavity with a first coil of the retraction instrument to secure the retraction instrument to the tissue, and retracting the tissue with the retraction instrument.

In aspects, engaging the tissue within the body cavity with the first coil includes rotating a handle of the retraction instrument in a first direction to rotate a shaft connected to the first coil to secure the first coil within the tissue. The method may include rotating a tube of the retraction instrument in a second direction that is opposite the first direction to secure a second coil extending from the tube in the tissue.

In some aspects, engaging the tissue within the body cavity with the first coil includes rotating a rod relative to a shaft of the retraction instrument to transition the first coil between a contracted configuration and an expanded configuration. The trailing end of the first coil may be secured to the shaft and a leading end of the first coil may be secured to a tip of the rod.

In another aspect of the present disclosure, a method of retracting tissue includes securing a first retraction coil to tissue, securing a second retraction coil to the tissue, and grasping an eyelet of the first retraction coil to retract the tissue.

In aspects, securing the first retraction coil to the tissue includes inserting a tip of the first retraction coil into the tissue with a coil of the first retraction coil disposed in a straight configuration and allowing the first retraction coil to transition to a coiled configuration which the first retraction coil is disposed within the tissue.

Further, to the extent consistent, any of the aspects described herein may be used in conjunction with any or all of the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a perspective view of a retraction instrument in accordance with the present disclosure with a coil of the retraction instrument in an undeployed, straight configuration;

FIG. 2 is a perspective view of the retraction instrument of FIG. 1 with the coil in a deployed, coiled configuration;

FIG. 3 is a cutaway view of a body cavity of a patient with the retraction instrument of FIG. 1 inserted into the body cavity;

FIG. 4 is a cutaway view of the body cavity of FIG. 3 with the retraction instrument secured to tissue within the body cavity;

FIG. 5 is a perspective view of another retraction instrument in accordance with the present disclosure with a handle in a retracted position and a coil in a contracted, undeployed configuration;

FIG. 6 is a perspective view of the retraction instrument of FIG. 5 with the handle in an extended position and the coil in the contracted, deployed configuration;

FIG. 7 is a perspective view of the retraction instrument of FIG. 6 with the coil in an extended, deployed configuration;

FIG. 8 is a perspective view of another retraction instrument in accordance with the present disclosure with a handle in an extended position such that a first coil is disposed distal of a second coil;

FIG. 9 is an enlarged, perspective view of a retraction coil in accordance with the present disclosure;

FIG. 10 is a perspective view of a plurality of retraction coils of FIG. 9 secured in tissue; and

FIG. 11 is a side view of another retraction coil in accordance with the present disclosure disposed in a straight configuration.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is closer to the clinician and the term “distal” refers to the portion of the device or component thereof that is farther from the clinician.

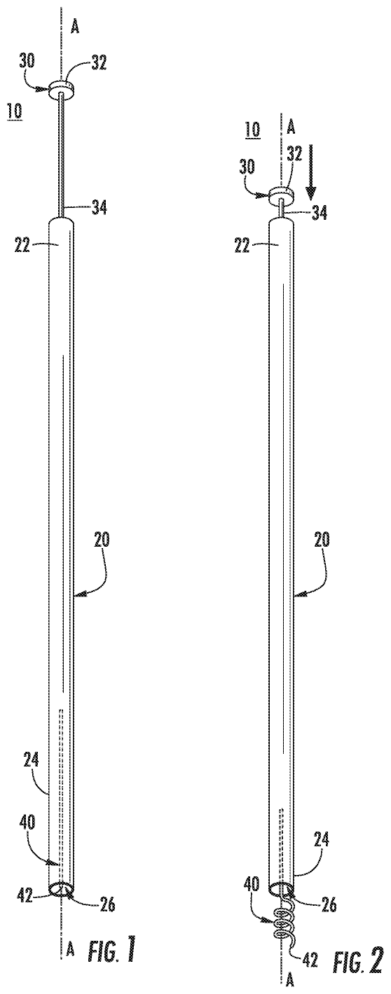

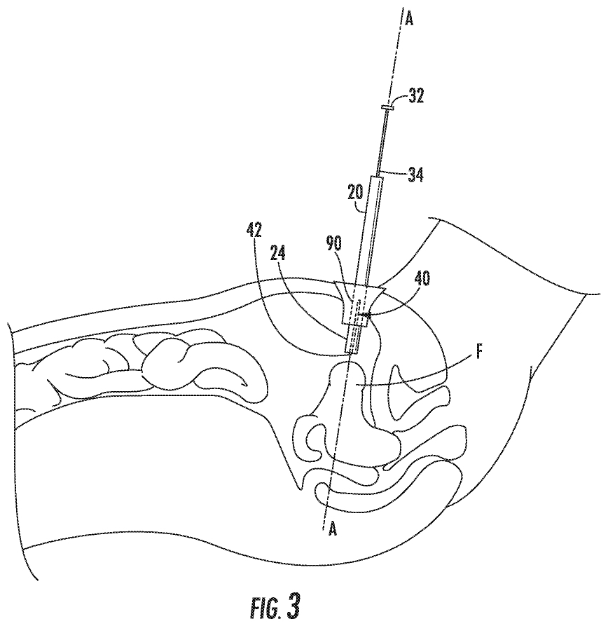

Referring now to FIGS. 1 and 2, a retraction instrument 10 is provided in accordance with the present disclosure. The retraction instrument 10 includes a tube 20, an actuator 30, and coil 40. The tube 20 includes a proximal portion 22 and a distal portion 24. The tube 20 is substantially cylindrical in shape and defines a longitudinal axis A-A and includes a passage 26 that is defined about the longitudinal axis A-A and extends through the tube 20 between the proximal and distal portions 22, 24.

The actuator 30 includes a handle 32 and a shaft 34 extending distally from the handle 32. The shaft 34 is fixed to the handle 32 such that the shaft 34 moves with the handle 32 as detailed below. The handle 32 is translatable along the longitudinal axis A-A between a retracted position (FIG. 1) and an extended position (FIG. 2) to translate the shaft 34 through the passage 26 of the tube 20. The handle 32 may be sized to prevent the handle 32 from passing through the passage 26. The handle 32 is also rotatable about the longitudinal axis A-A such that the shaft 34 is rotated about the longitudinal axis A-A. In embodiments, the handle 32 is fully retractable such that the entire shaft 34 is withdrawn from the passage 26 of the tube 20.

The coil 40 is secured to the shaft 34 and is extendable from the distal portion 24 of the tube 20. The coil 40 is made from a super elastic material that has a straight configuration (FIG. 1) and a coiled configuration (FIG. 2). The coil 40 has a tip 42 that may be sharpened to penetrate into tissue. The coil 40 may be constructed from a shape memory alloy, e.g., Nitnol.

More particularly, shape memory alloys (SMAs) are a family of alloys having anthropomorphic qualities of memory and trainability and are particularly well suited for use with medical instruments. SMAs have been applied to such items as actuators for control systems, steerable catheters and clamps. One of the most common SMAs is Nitinol which can retain shape memories for two different physical configurations and changes shape as a function of temperature. SMAs have also been developed based on copper, zinc and aluminum and have similar shape memory retaining features.

SMAs undergo a crystalline phase transition upon applied temperature and/or stress variations. A particularly useful attribute of SMAs is that after it is deformed by temperature/stress, it can completely recover its original shape on being returned to the original temperature. The ability of an alloy to possess shape memory is a result of the fact that the alloy undergoes a reversible transformation from an austenite state to a martensite state with a change in temperature (or stress-induced condition). This transformation is referred to as a thermoelastic martensite transformation.

Under normal conditions, the thermoelastic martensite transformation occurs over a temperature range which varies with the composition of the alloy, itself, and the type of thermal-mechanical processing by which it was manufactured. In other words, the temperature at which a shape is “memorized” by an SMA is a function of the temperature at which the martensite and austenite crystals form in that particular alloy. For example, Nitinol alloys can be fabricated so that the shape memory effect will occur over a wide range of temperatures, e.g., −270° to +100° Celsius.

Many SMAs are also known to display stress-induced martensite (SIM) which occurs when the alloy is deformed from its original austenite state to a martensite state by subjecting the alloy to a stress condition. For example and with respect to FIGS. 1 and 2 of the present disclosure, coil 40 is generally bent or “coiled” when disposed in its original or austenite state (see FIG. 2). When coil 40 is inserted into the tube 20, coil 40 is deformed, i.e., straightened, into a stress-induced martensite state enabling the user to more easily navigate the tube 20 through tight body cavities and passageways to access tissue.

With reference to FIG. 3, the distal portion 24 of the instrument 10 is inserted into a body cavity of a patient through an opening with the handle 32 in the retracted position such that the tip 42 of the coil 40 remains within tube 20 (or extends slightly beyond the distal portion 24 of the tube 20). The opening may be a natural opening or may be an incision. A port 90 may be inserted within the opening to maintain the opening and provide a channel for the instrument 10 to be inserted into the body cavity. When the instrument 10 is inserted into the body cavity, the coil 40 is in the straight configuration (FIG. 1).

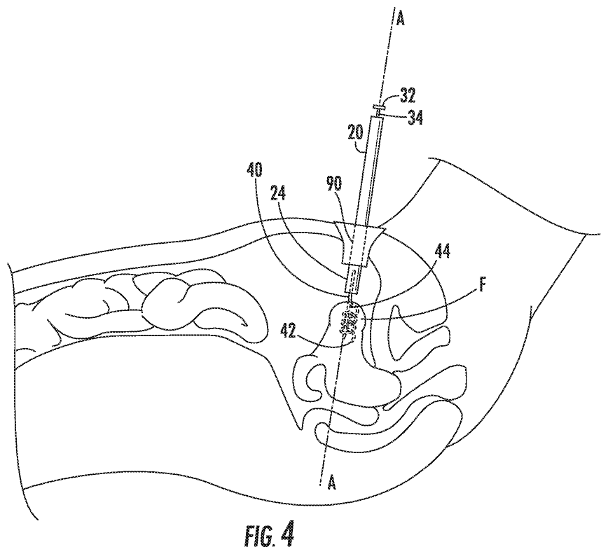

Referring to FIG. 4, with the distal portion 24 disposed within the body cavity, the distal portion 24 is positioned adjacent a fibroid F with the longitudinal axis A-A generally aligned with the fibroid F. When properly aligned, the handle 32 is translated to extend the coil 40 to penetrate the fibroid F. The tip 42 may be sharpened to facilitate penetration of the coil 40 into the fibroid F.

As the coil 40 penetrates into the fibroid F, a portion 44 of the coil 40 that is disposed within the fibroid F transitions from the straight configuration to the coiled configuration to secure the coil 40 to the fibroid F. The portion 44 of the coil 40 secures the shaft 34 of the actuator 30 to the fibroid F. With the fibroid F secured, another instrument (not shown) may be used to dissect the fibroid F from surrounding tissue such that the fibroid F can be removed from the body cavity. In embodiments, the handle 32 and/or the tube 20 of the surgical instrument 10 may be manipulated to aid in the dissection of the fibroid F.

With the fibroid F dissected from surrounding tissue, the handle 32 is retracted to draw the fibroid F towards the distal portion 24 of the tube 20. The fibroid F may be drawn into the passage 26 of the tube 20 and the entire instrument 10 may be withdrawn from the body cavity to remove the fibroid F from the body cavity.

In embodiments, a morcellator may be utilized to reduce the size of the fibroid F to allow the fibroid F to be removed from the access point (incision or natural body orifice) of the body cavity. In some embodiments, a portion of the fibroid F may be removed through the tube 20 by fully retracting the handle 32 such that the portion of the fibroid F is removed from the body cavity through the tube 20. The coil 40 and the shaft 34 may then be reinserted through the tube 20 to engage another portion of the fibroid F to remove the other portion of the fibroid F through the tube 20. This process may be repeated until the entire fibroid F is removed from the body cavity.

With reference to FIGS. 5-7, another retraction instrument 110 is shown in accordance with the present disclosure. The retraction instrument 110 includes a tube 120, an actuator 130, and a coil 140. The tube 120 includes a proximal portion 122 and a distal portion 124. The tube 120 is substantially cylindrical in shape and defines a longitudinal axis A-A. The tube 120 includes a passage 126 defined within tube 120 between the proximal and distal portions 122, 124.

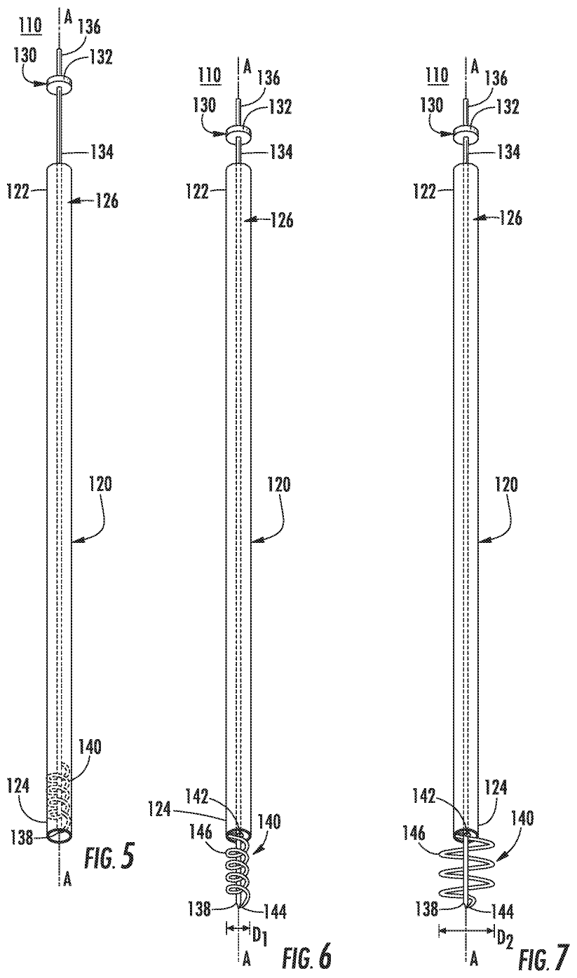

The actuator 130 includes a handle 132 and a shaft 134 extending distally from the handle 132. The shaft 134 is fixed to the handle 132 such that the shaft 134 moves with the handle 132. Specifically, the handle 132 is translatable along the longitudinal axis A-A between a retracted position (FIG. 5) and an extended position (FIG. 6) to translate the shaft 134 through the passage 126 of the tube 120. The handle 132 may be sized to prevent the handle 132 from passing through the passage 126. The handle 132 is also rotatable about the longitudinal axis A-A such that the shaft 134 correspondingly rotates about the longitudinal axis A-A. In embodiments, the handle 132 is retractable such that the entire shaft 134 may be withdrawn from the passage 126 of the tube 120. The actuator 130 also includes a rod 136 that passes through the handle 132 and the shaft 134 to a tip 138. The rod 136 is rotatable independent of the shaft 134. The tip 138 is fixed to the rod 136 such that the tip 138 rotates with the rod 136. In some embodiments, the rod 136 may be translatable relative to the shaft 134 such that the tip 138 extends away from a distal end of the shaft 134. Alternatively, the rod 136 may be longitudinally fixed relative to the shaft 134.

The coil 140 includes a trailing end 142, a leading end 144, and winds 146 disposed between the trailing and leading ends 142, 144. The trailing end 142 is secured to an outer surface of the shaft 134 and the leading end 144 is secured to the tip 138 of the rod 136. The coil 140 has a helical shape such that the winds 146 of the coil 140 wrap about the shaft 134. The coil 140 may be formed of a metal, e.g., surgical steel or spring steel, and may be formed of a super elastic material, e.g., Nitnol.

With particular reference to FIG. 5, the coil 140 when disposed in an undeployed, retracted position, the leading end 144 of the coil 140 is disposed within the distal portion 124 of the tube 120. In the retracted position, the tip 138 may extend distally beyond the distal portion 124 of the tube 120.

Referring to FIG. 6, when the coil 140 is extended by actuation of actuator 130, the coil 140 is positioned distal of the distal portion 124 of the tube 120 such that some or all of the winds 146 are distal of the distal portion 124 of the tube 120. To transition the coil 140 from the retracted position to the extended position, the tube 120 is translated proximally relative to the shaft 134 and/or the handle 132 is translated distally to translate the shaft 134 distally relative to the tube 120. When the coil 140 is in the extended position, the trailing end 142 of the coil 140 may be disposed within the distal portion 124 of the tube 120 or may be positioned distal of the distal portion 124 of the tube 120.

When the coil 140 is in the retracted position and the extended position as shown in FIGS. 5 and 6, the coil 140 is in a contracted configuration such that the winds 146 have a first diameter D₁. The first diameter D₁ may be greater than a diameter of the distal portion 124 of the tube 120 or may be less than the diameter of the distal portion 124 of the tube 120. When the coil 140 is in the retracted position and the first diameter D₁ is greater than or equal to the diameter of the distal portion 124, the winds 146 of the coil 140 engage an inner surface of the tube 120. When the winds 146 of the coil 140 engage the inner surface of the tube 120, the coils 146 resist longitudinal translation relative to the tube 120 such that the coils longitudinally fix the shaft 134 relative to the tube 120 absent an external force to translate the shaft 134 relative to the tube 120. The coil 140 may have a fully contracted configuration (not shown) in which the winds 146 of the coil 140 are wrapped about and in contact with the shaft 134. The coil 140 has an expanded configuration in which the winds 146 have a second diameter D₂ that is greater than the first diameter D₁ as shown in FIG. 7.

Referring to FIGS. 5-7, the coil 140 is transitionable between the contracted configuration and the expanded configuration by rotating the rod 136 relative to the shaft 134. As detailed above, a portion of the rod 136 may extend proximally through the handle 132 such that the rod 136 may be rotated relative to the shaft 134 by rotating the portion of the rod 136 and holding the handle 132 stationary, holding the portion of the rod 136 stationary and rotating the handle 132, or by rotating the portion of the rod 136 and the handle 132 in opposite directions. When the rod 136 is rotated relative to the shaft 134, the tip 138 of the rod 136 rotates the leading end 144 of the coil 140 relative to the trailing end 142 of the coil 140. As the leading end 144 is rotated towards the trailing end 142, e.g., counter-clockwise as shown, the winds 146 are expanded or driven outward to increase the diameter of the winds 146. When the leading end 144 is rotated away from the trailing end 142, e.g., clockwise as shown, the loops 146 are contracted towards the fully contracted configuration to decrease the diameter of the winds 146.

In use, the retraction instrument 110 is inserted into a body cavity with the coil 140 in the retracted configuration in a similar manner to the retraction instrument 10 detailed above. The tip 138 of the rod 136 is positioned adjacent a fibroid F with the longitudinal axis A-A of the retraction instrument 110 passing through the fibroid F. The coil 140 is then transitioned into the extended position by pushing the handle 132 distally such that the tip 138 is driven into the fibroid F with the coil 140 in a contracted configuration. The tip 138 may be sharpened to aid in penetration of the fibroid F.

With the tip 138 disposed within the fibroid F, the rod 136 is rotated relative to the shaft 134 to transition the coil 140 towards the expanded configuration. As the coil 140 transitions towards the expanded configuration, the winds 146 of the coil 140 expand into and secure the shaft 134 and the rod 136 to the fibroid F. With the fibroid F secured to the shaft 134 and the rod 136, the retraction instrument 110 can be manipulated to dissect and remove the fibroid F from the body cavity in a manner similar to the retraction instrument 10 detailed above.

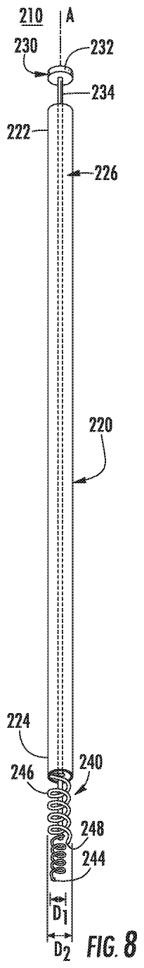

With reference to FIG. 8, another retraction instrument 210 is disclosed in accordance with the present disclosure. The retraction instrument 210 includes a tube 220, an actuator 230, and a coil assembly 240. The tube 220 includes a proximal portion 222 and a distal portion 224. The tube 220 is substantially cylindrical in shape and defines a longitudinal axis A-A. The tube 220 includes a passage 226 that is defined about the longitudinal axis A-A and that extends through the tube 220 between the proximal and distal portions 222, 224.

The actuator 230 includes a handle 232 and a shaft 234 extending distally from the handle 232. The shaft 234 is fixed to the handle 232 such that the shaft 234 moves with the handle 232. Specifically, the handle 232 is translatable along the longitudinal axis A-A between a retracted position (not shown) and an extended position to translate the shaft 234 through the passage 226 of the tube 220. The handle 232 may be sized to prevent the handle 232 from passing through the passage 226. The handle 232 is also rotatable about the longitudinal axis A-A such that the shaft 234 may be rotated about the longitudinal axis A-A. In embodiments, the handle 232 is retractable such that the entire shaft 234 may be withdrawn from the passage 226 of the tube 220.

The coil assembly 240 includes a first coil 242 and a second coil 246. The first coil 242 is fixed to and extends distally from the shaft 234 to a first tip 244. The first coil 242 is a helical coil that is wound in a first direction, e.g., clockwise. The first coil 242 may be have a constant diameter, e.g., first diameter D₁, or may have a constantly decreasing diameter from the first diameter D₁ such that the first tip 244 is disposed along the longitudinal axis A-A. The first coil 242 cooperates with rotation of the handle 242. The second coil 246 is fixed to and extends distally from the distal portion 224 of the tube 220 to a second tip 248. The second coil 246 is a helical coil that is wound in a second direction opposite the first direction, e.g., counter-clockwise. The second coil 246 has a constant diameter, e.g., second diameter D₂, that is greater than the first diameter D₁. The second diameter D₂ is large enough to allow the shaft 234 and the first coil 242 to pass through the second coil 246. The second coil 246 cooperates with rotation of the tube 220. The first and second coils 242, 244 may be rigid or may be at least partially flexible. The first and second tips 244, 248 may be sharpened to facilitate penetration of tissue as detailed below.

When the handle 232 is in the retracted position, the first coil 242 is disposed within the distal portion 242 of the tube 220. In the retracted position, the first tip 244 of the first coil 242 is disposed within the distal portion 242 or may be positioned distal of the distal portion 242. When the handle 232 is in the extended position, the first coil 242 is positioned distal of the second tip 248 of the second coil 248. In the extended position, at least the first tip 244 of the first coil 242 is positioned distal of the second tip 248. In the extended position, the entire first coil 242 may be positioned distal of the second tip 248.

In use, the retraction instrument 210 is inserted into a body cavity with the first coil 242 retracted within the distal portion 224 of the tube 220 in a similar manner to the retraction instrument 10 detailed above. The second tip 248 of the second coil 248 is positioned within the body cavity adjacent a fibroid F with the longitudinal axis A-A of the retraction instrument 210 passing through the fibroid F. The handle 232 is then pushed in a direction towards the fibroid F to extend the first coil 242 from within the tube 220 such that the first tip 244 engages the fibroid F. The handle 232 is then rotated in the first direction, e.g., clockwise, to drive the first tip 244, and thus the first coil 242, into the fibroid F. With the first coil 242 disposed within the fibroid F, the tube 220 is advanced towards the fibroid F until the second tip 248 of the second coil 246 engages the fibroid F. The tube 220 is then rotated in the second direction opposite the first direction, e.g., counter-clockwise, to drive the second tip 248, and thus the second coil 246, into the fibroid F. With the first and second coils 242, 246 disposed within the fibroid F, the retraction instrument 210 can be manipulated to dissect and remove the fibroid F from the body cavity in a manner similar to the retraction instrument 10 detailed above.

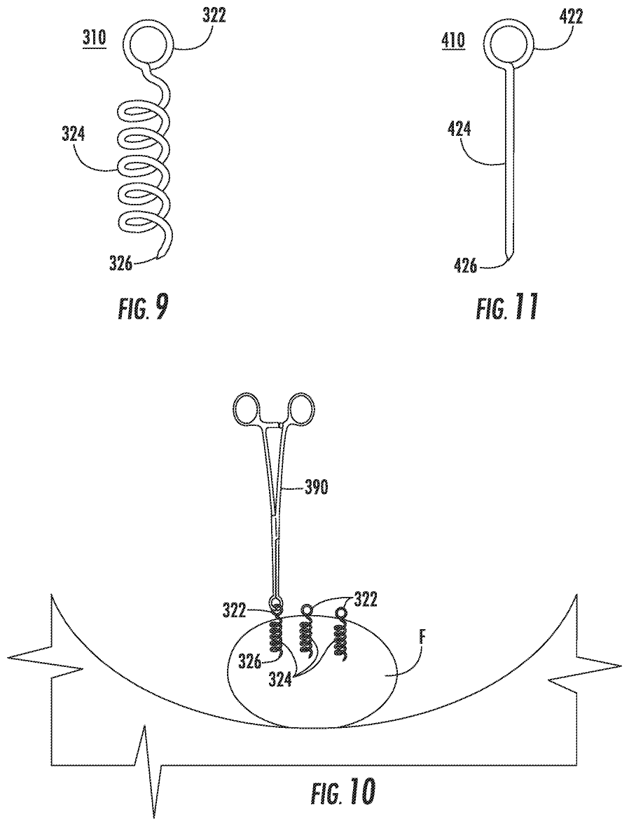

With reference to FIGS. 9 and 10, a retraction coil 310 is disclosed in accordance with the present disclosure. The retraction coil 310 includes an eyelet 322, a shaft 324, and a tip 326. The shaft 324 extends distally from the eyelet 322 to the tip 326. The shaft 324 is a helical coil. The tip 326 may be sharpened to aid in penetrating tissue as detailed below.

In use, one or more retraction coils 310 are inserted into a fibroid F to provide an attachment point for a surgical instrument 390. Specifically, the eyelet 322 of the retraction coil 310 may be grasped by a surgical instrument 390, e.g., a grasper, and positioned adjacent the fibroid F with the tip 326 of the retraction coil 310 engaged with the fibroid F. The surgical instrument 390 is then rotated to drive the tip 326, and thus the coil 324, into the fibroid F. The surgical instrument 390 may be used to drive additional retraction coils 310 into the fibroid F.

With the retraction coil 310 secured in the fibroid F, the surgical instrument 390, or another surgical instrument (not shown), may grasp or be passed through the eyelet 322 to manipulate the fibroid F such that the fibroid F may be dissected from tissue and/or removed from a body cavity.

With reference to FIG. 11, another retraction coil 410 is disclosed in accordance with the present disclosure. The retraction coil 410 includes an eyelet 422, a shaft 424, and a tip 426. The shaft 424 extends distally from the eyelet 422 to the tip 426. The shaft 424 is made of a super elastic material, e.g., Nitnol, and has a straight configuration and a coiled configuration similar to coil 40 detailed above.

In use, the retraction coil 410 is inserted into a fibroid F by driving the tip 426 into the fibroid F with the shaft 424 in the straight configuration. The tip 426 may be sharpened to aid in penetration of tissue. When the shaft 424 is disposed within the fibroid F, the shaft 424 transitions to the coiled configuration to secure the retraction coil 410 into the fibroid F. A surgical instrument such as surgical instrument 390 (FIG. 10) may be used to drive the shaft 424 into the fibroid F. When the retraction coil 410 is secured in the fibroid F, the surgical instrument 390, or another surgical instrument (not shown), may grasp or be passed through the eyelet 422 to manipulate the fibroid F such that the fibroid F may be dissected from tissue and/or removed from a body cavity.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A retraction instrument, comprising:
a tube defining a passage therethrough;
an actuator having a handle, the handle having a shaft extending distally therefrom through the passage of the tube; and
a first coil disposed adjacent a distal end of the shaft, the first coil configured to secure the shaft to tissue.

2. The retraction instrument according to claim 1 wherein the first coil extends distally from the distal end of the shaft.

3. The retraction instrument according to claim 2 wherein the first coil includes a straight configuration in which the first coil is substantially linear and a coiled configuration in which the first coil is substantially non-linear and configured to secure the first coil within tissue.

4. The retraction instrument according to claim 3 wherein the first coil is configured to transition from the straight configuration to the coiled configuration as the first coil is inserted into tissue.

5. The retraction instrument according to claim 2 wherein the shaft has a retracted position in which the first coil is disposed within the tube and an extended position in which the first coil is positioned distal of the tube.

6. The retraction instrument according to claim 2 further comprising a second coil secured to a distal portion of the tube and extending distally from the distal portion of the tube.

7. The retraction instrument according to claim 6 wherein the first coil is wound in a first direction and the second coil is wound in a second, opposite direction.

8. The retraction instrument according to claim 6 wherein the first coil has first diameter and the second coil has a second diameter which is greater than the first diameter.

9. The retraction instrument according to claim 6 wherein the first coil has a substantially constant diameter.

10. The retraction instrument according to claim 1 further comprising a rod extending through the actuator to a tip disposed distal of the distal end of the shaft.

11. The retraction instrument according to claim 10 wherein the first coil includes a leading end secured to the tip, a trailing end secured to the shaft, and winds about the shaft between the leading and trailing ends.

12. The retraction instrument according to claim 11 wherein the first coil has a contracted configuration in which the first coil defines a first diameter and an expanded configuration in which the first coil defines a second diameter greater than the first diameter.

13. The retraction instrument according to claim 12 wherein the rod is rotatable relative to the shaft to transition the first coil between the contracted and expanded configurations.

14. The retraction instrument according to claim 12 wherein the first diameter is less than a diameter of the passage.

15. A method of retracting tissue within a body cavity, the method comprising:
inserting a retraction instrument into the body cavity of a patient;
engaging tissue within the body cavity with a first coil of the retraction instrument to secure the retraction instrument to the tissue; and
retracting the tissue with the retraction instrument.

16. The method according to claim 15 wherein engaging the tissue within the body cavity with the first coil includes inserting the first coil into the tissue in a straight configuration in which the first coil is substantially linear and allowing the first coil to transition to a second configuration while disposed within the tissue.

17. The method according to claim 15 wherein engaging the tissue within the body cavity with the first coil includes rotating a handle of the retraction instrument in a first direction to rotate a shaft connected to the first coil to secure the first coil within the tissue, and the method further comprising rotating a tube of the retraction instrument in a second direction, opposite the first direction, to secure a second coil extending from the tube in the tissue.

18. The method according to claim 15 wherein engaging the tissue within the body cavity with the first coil includes rotating a rod relative to a shaft of the retraction instrument to transition the first coil between a contracted configuration and an expanded configuration, a trailing end of the first coil secured to the shaft and a leading end of the first coil secured to a tip of the rod.

19. A method of retracting tissue within a body cavity, the method comprising:
securing a first retraction coil to tissue;
securing a second retraction coil to the tissue; and
grasping an eyelet of the first retraction coil to retract the tissue.

20. The method according to claim 19, wherein securing the first retraction coil to tissue includes inserting a tip of the first retraction coil into the tissue with a coil of the first retraction coil disposed in a straight configuration and allowing the first retraction coil to transition to a coiled configuration while the first retraction coil is disposed within the tissue.
